(19) United States
(12) Patent Application Publication
(10) Pub. No.: US 2025/0283837 A1
(43) Pub. Date: Sep. 11, 2025

(54) Method and Device for Measuring Edible Rate of Pomelo Fruit Based on Machine Vision and X-Ray Imaging

(71) Applicant: Institute of Facility Agriculture, Guangdong Academy of Agricultural Sciences, Guangzhou (CN)

(72) Inventors: Sai Xu, Guangzhou (CN); Huazhong Lu, Guangzhou (CN); Xin Liang, Guangzhou (CN)

(21) Appl. No.: 19/097,904

(22) Filed: Apr. 2, 2025

Related U.S. Application Data
(63) Continuation of application No. PCT/CN2024/106108, filed on Jul. 18, 2024.

(30) Foreign Application Priority Data
Mar. 5, 2024 (CN) 202410245655.9

(51) Int. Cl. G01N 23/04; G01N 33/02; G06T 5/80; G06T 7/00; G06T 7/136; G06T 7/194; G06T 7/62; G06T 7/90; G06T 17/00

(52) U.S. Cl. CPC G01N 23/04; G01N 33/025; G06T 5/80; G06T 7/0004; G06T 7/136; G06T 7/194; G06T 7/62; G06T 7/90; G06T 17/00; G01N 2223/3308; G01N 2223/401; G01N 2223/423; G01N 2223/618; G06T 2207/10024; G06T 2207/10116; G06T 2207/30128

(57) ABSTRACT

The present disclosure discloses a method and device for measuring an edible rate of a pomelo fruit based on machine vision and X-ray imaging. The method include: step 1, collecting an appearance image of the pomelo fruit; step 2, performing image segmentation on the appearance image of the pomelo fruit and removing a background image of the pomelo fruit, step 3, slicing the appearance image of the pomelo fruit, determining an endpoint on each slice of the pomelo fruit, and determining a slice outline using a B-spline curve interpolation fitting method based on the endpoint; step 4, establishing a fresh model thickness image by projecting a flesh area in a three-dimensional model of the pomelo fruit along a X-ray imaging direction; step 5, a value of each point in the flesh model thickness image representing a flesh thickness of the corresponding point along the X-ray imaging direction.

### CROSS-REFERENCE TO RELATED APPLICATIONS

The present disclosure claims the priority of Invention Application No. PCT/CN2024/106108, filed on Jul. 18, 2024, the entire contents of which are hereby incorporated by reference.

### FIELD

The present disclosure relates to image processing technology, and more particularly, to a method and device for measuring an edible rate of a pomelo fruit based on machine vision and X-ray imaging.

### BACKGROUND

Quality inspection and grading of agricultural products are important to the industry to improve economic efficiency and upgrade development. An edible rate is a key internal quality indicator of a pomelo fruit, which represents a portion of flesh content in the pomelo fruit. However, due to inherent biological variations in fruit growth, which means that the fruit shapes are irregular, the peel is thick and the fruit is large, practical detection of the edible rate faces challenges in accuracy and efficiency. At present, there are few reports on non-destructive detection methods for the edible rate of the pomelo fruit, and the non-destructive testing methods are not effectively applied in actual production. In order to meet the accurate and rapid non-destructive detection requirements of the pomelo industry for the edible rate, the present disclosure proposes a measuring algorithm for the edible rate of the pomelo fruit based on machine vision and X-ray imaging.

### SUMMARY

In view of this, the present disclosure provides a method and device for measuring an edible rate of a pomelo fruit based on machine vision and X-ray imaging.

In order to achieve the above objectives, the present disclosure adopts the following technical solution:

A method for measuring an edible rate of a pomelo fruit based on machine vision and X-ray imaging, including the following steps:
Step 1, collecting an appearance image of a pomelo fruit;
Step 2, performing image segmentation on the appearance image of the pomelo fruit and removing a background image of the pomelo fruit;
Step 3, slicing the appearance image of the pomelo fruit, determining an endpoint on each slice, and determining a slice outline using a B-spline curve interpolation fitting method based on the endpoint;
Step 4, based on the slice outline, establishing a fresh model thickness image by projecting a flesh area in a three-dimensional model of the pomelo fruit along an X-ray imaging direction, with a transverse diameter and a longitudinal diameter of the slice as an x-axis direction and a y-axis direction, respectively;
Step 5, a value of each point in the flesh model thickness image representing a flesh thickness of the corresponding point along the X-ray imaging direction.

In an embodiment, the method for measuring an edible rate of a pomelo fruit includes the following steps:
step 1, collecting the appearance image of the pomelo fruit and obtaining an X-ray image and a weight of the pomelo fruit from a direction perpendicular to a longitudinal diameter of the pomelo fruit;
step 2, performing the image segmentation on the appearance image of the pomelo fruit and removing the background image of the pomelo fruit;
step 3, slicing the appearance image of the pomelo fruit, determining the endpoint on the slice, and determining the slice outline using the B-spline curve interpolation fitting method based on the endpoint obtain an outer contour three-dimensional model of the pomelo fruit;
step 4, based on the slice outline, obtaining the flesh area of in the outer contour three-dimensional model of the pomelo fruit by combining the outer contour three-dimensional model of the pomelo fruit and the X-ray image, with the transverse diameter and the longitudinal diameter as the x-axis direction and the y-axis direction, respectively; establishing a flesh model thickness image of the flesh area in the outer contour three-dimensional model by projecting the flesh area in the outer contour three-dimensional model of the pomelo fruit along the X-ray imaging direction;
step 5, the value of each point in the flesh model thickness image representing the flesh thickness of the corresponding point along the X-ray imaging direction;
using a fitting algorithm to calculate a fitting function between a thickness of a certain point in the flesh model thickness image and a grayscale of the certain point in a X-ray grayscale image;
using the fitting function to correct the X-ray grayscale image to obtain a corrected X-ray grayscale image.
calculating the edible rate based on the weight of the pomelo fruit, the fitting function, the grayscale of each point in the flesh in the corrected X-ray grayscale image, a flesh density of the pomelo fruit, and a width and a height of the X-ray image of the pomelo fruit.

In an embodiment, the width and the height of the X-ray image of the pomelo fruit is obtained according to the corrected X-ray grayscale image.

In an embodiment, before the collecting an appearance image of a pomelo fruit, a Zhang's calibration is performed using a RGB camera, and a distortion correction is performed based on an intrinsic parameter, an extrinsic parameters, and a distortion coefficient of the camera.

In an embodiment, the distortion correction is performed according the following geometric relationship to obtain an actual width D of the pomelo fruit

$$D = \frac{w}{d} \sqrt{{w}^{2} + {d}^{2}} \qquad ( 1 )$$

wherein w is a pixel width of the pomelo fruit in the appearance image; d is a distance between the pomelo fruit and the camera, and a corrected distance d between another pomelo fruit and the camera is defined as:

$$d = {d}_{m} + \frac{0.5 \times {d}_{m} ( {w}_{m} - w )}{f} \qquad ( 2 )$$

wherein wₘ is a distance between a medium-sized pomelo fruit and the camera, dₘ is a pixel width of the medium-sized pomelo fruit, and f is a focal length of the camera.

In an embodiment, in the step 2, a threshold segmentation method is used to capture a pomelo fruit area, including:
converting the appearance image of the pomelo fruit from a RGB color space to a HSI color space using a dual channel fusion processing method;
extracting a hue H channel and a brightness I channel;
performing threshold segmentation on the hue H channel and the brightness I channel respectively; and
superimposing the hue H channel and the brightness I channel after the threshold segmentation to form the pomelo fruit area.

In an embodiment, in the step 4, pixel thicknesses t₁ and t₂ of a peel at both ends of each slice is obtained in the X-ray image, and an average of the pixel thicknesses t₁ and t₂ is taken as an average pixel thickness t of the pomelo fruit slice; the slice outline in contracted inward for the pixel thickness t, which indicates that a polar diameter of each point on the outer contour is decreased by t to obtain a new contour line which is used as a flesh outer contour in the slice.

The present disclosure further provides a device for measuring an edible rate of a pomelo fruit based on machine vision and X-ray imaging using the above method, wherein the device includes a conveyor belt, a RGB camera, a camera bracket, and a X-ray detection apparatus; the RGB camera is fixed on the camera bracket for adjusting a position of the pomelo fruit; the conveyor belt is used for transporting the pomelo fruit; and the X-ray detection apparatus is equipped with a radiation source for X-ray detection of the pomelo fruit.

Based on the above technical solution, it can be seen that compared with the existing technology, the method and the device for measuring an edible rate of a pomelo fruit based on machine vision and X-ray imaging provided in the present disclosure has the following beneficial effects:

Firstly, by using the conveyor belt, the edible rate of the pomelo fruit can be quickly and efficiently detected as the pomelo fruit moves.

Secondly, only one X-ray image is required to be captured for one pomelo fruit, allowing the device to have a simple structure and carries out the detection rapidly.

Thirdly, the X-ray imaging technology can obtain internal image information of the pomelo fruit, having a precise detection effect.

In addition, in the present disclosure, the three-dimensional model of the pomelo fruit is reconstructed based on the B-spline to measure the volume of the pomelo fruit, while fitting the thickness of the flesh and the X-ray grayscale to measure the edible rate. Through this method, there is no need for traditional techniques requiring X-CT multi-directional scanning imaging, which can effectively reduce equipment costs. At the same time, the method of the present disclosure has a high accuracy and a strong practicality.

### BRIEF DESCRIPTION OF THE DRAWINGS

To describe the technical solutions in the embodiments of the present disclosure more clearly, the following briefly introduces the accompanying drawings required for describing the embodiments or the prior art. Apparently, the accompanying drawings in the following description show merely some embodiments of the present disclosure, and persons of ordinary skill in the art may still derive other drawings from these accompanying drawings without creative efforts.

FIG. 1 is a schematic view of a device according to an embodiment of the present disclosure;

FIG. 2 is a schematic diagram showing image segmentation and matching;

FIG. 3a is a schematic diagram showing a slice integration method as an implementation form;

FIG. 3b is a schematic diagram of a slicing process during an establishment process of a three-dimensional model of a pomelo fruit according to an embodiment of the present disclosure;

FIG. 3c is a schematic diagram of extracting endpoints from three appearance images during the establishment process of the three-dimensional model of the pomelo fruit according to an embodiment of the present disclosure;

FIG. 3d is a schematic diagram of an outer contour three-dimensional model of the pomelo fruit obtained by a measurement method based on B-spline curve fitting contour during the establishment process of the three-dimensional model of the pomelo fruit according to an embodiment of the present disclosure;

FIG. 4a is a schematic diagram of a pixel thickness of a peel at both ends of each slice in a X-ray image of a three-dimensional modeling method (3DMM);

FIG. 4b is a schematic diagram of a flesh contour of in the slice obtained by the 3DMM;

FIG. 4c is a schematic diagram of the three-dimensional model of the pomelo fruit with the flesh in the 3DMM;

FIG. 5 shows a fitted slice of the pomelo fruit;

FIG. 6 shows a fitted flesh X-ray image and a flesh model thickness image;

FIG. 7a shows a relationship between a measured flesh volume and an actual flesh weight obtained by the 3DMM;

FIG. 7b shows a relationship between the measured flesh volume and the actual flesh weight obtained by a grayscale and thickness fitting method (GTFM);

FIG. 8a shows edible rate measurement results obtained by the 3DMM;

FIG. 8b shows the edible rate measurement results obtained by the 3DMM.

### DESCRIPTION OF REFERENCE NUMERALS

1, Camera bracket; 2, RGB camera; 3, Conveyor belt; 4, Pomelo fruit; 5, X-ray detection apparatus; 6, Radiation source; 7, Conveyor belt direction.

### DETAILED DESCRIPTION

In order to make the objectives, features, and advantages of the present disclosure more obvious and understandable, the technical solutions in the embodiments of the present disclosure will be clearly and completely described below in conjunction with the accompanying drawings of the embodiments of the present disclosure. It is apparent that the embodiments described below are only a part of the embodiments of the present disclosure, not all of them. Based on the embodiments of the present disclosure, all other embodiments obtained by those skilled in the art without creative effort fall within the scope of protection of the present disclosure.

Below, the technical solutions in the embodiments of the present disclosure will be clearly and completely described in conjunction with the accompanying drawings. Obviously, the described embodiments are only a part of the embodiments of the present disclosure, not all of them. Based on the embodiments of the present disclosure, all other embodiments obtained by ordinary skilled persons in the art without creative labor are within the scope of protection of the present disclosure.

The present embodiment discloses a method for measuring an edible rate of a pomelo fruit based on machine vision and X-ray imaging. The method of this embodiment is divided into the following parts for sequential discussion: Part I, Construction of pomelo fruit detection platform; Part II, Image acquisition and processing; Part III, Volume algorithm; Part IV, Edible rate algorithm.

### Part I, Construction of Pomelo Fruit Detection Platform

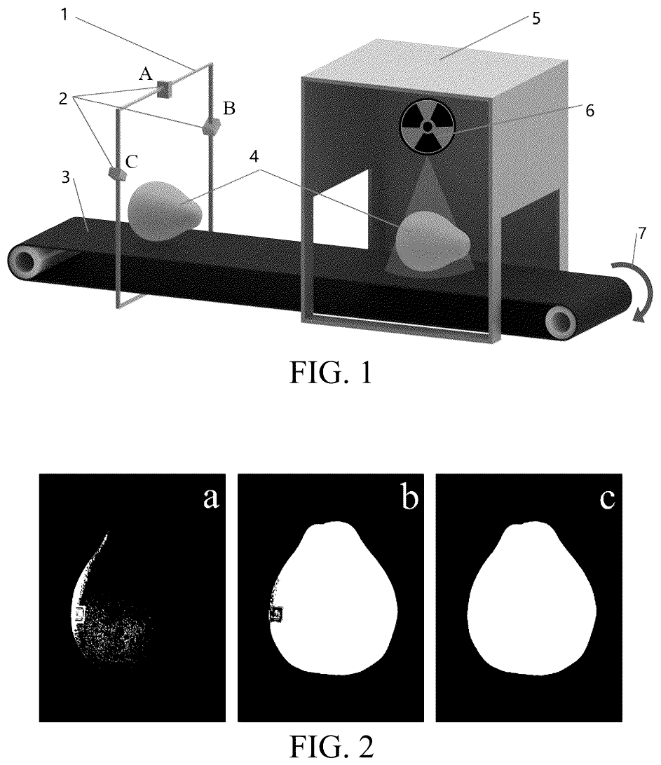

As shown in FIG. 1, a camera bracket 1, a RGB camera 2, a conveyor belt 3 with a direction 7, a pomelo fruit 4, a X-ray detection apparatus 5, and a radiation source 6 are included. The pomelo fruit 4 enters from a left end of the conveyor belt 3 and an appearance image of the pomelo fruit is captured. The appearance image of the pomelo fruit is obtained using three RGB cameras 2 with 8-mm lenses, wherein a RGB camera A vertically faces downwards to capture an upper part of the pomelo fruit, a camera B and a camera C both have an angle of 30° with respect to a horizontal plane to respectively capture left and right sides of the pomelo fruit. To reduce external light interference, the appearance image is captured in a dark box. To fix the posture more uniformly, the pomelo fruit is placed flat on a specially designed circular tray. After the appearance image of the pomelo fruit is obtained, the pomelo fruit is transported along the conveyor belt to the X-ray detection apparatus 5. To ensure a more accurate fit between an X-ray image and the appearance image, the posture of the pomelo fruit is required to be consistent with that when the appearance image is captured.

### Part II, Image Acquisition And Processing

Before collecting image data, a Zhang's calibration is performed on the RGB camera and a distortion correction is performed based on a camera intrinsic parameter, a camera extrinsic parameter, and a distortion coefficient. The distortion correction can be performed based on the geometric relationship to obtain an actual width of the pomelo fruit:

$$D = \frac{w}{d} \sqrt{{w}^{2} + {d}^{2}} \qquad ( 1 )$$

Wherein w is a pixel width of the pomelo fruit in the image, and d is a distance between the pomelo fruit and the camera.

An actual distance error between the object and the camera has a certain impact on the measurement accuracy. Due to the large size of the pomelo fruits, the distance between different pomelo fruits and the camera may vary greatly depending on the size of the pomelo. To minimize the distance errors as much as possible, it is necessary to calibrate the distance between the camera and the pomelo fruit based on the size of the pomelo fruit. Due to the approximate circular shape of the pomelo fruit in a transverse direction, a vertical height change caused by the size of the pomelo fruit can be represented by a horizontal width change of the pomelo fruit. Therefore, using a distance between a medium-sized pomelo fruit and the camera as a reference, a corrected distance d between another pomelo fruit and the camera can be defined as:

$$d = {d}_{m} + \frac{0.5 \times {d}_{m} ( {w}_{m} - w )}{f} \qquad ( 2 )$$

Wherein wₘ is the distance between the medium-sized pomelo fruit and the camera, dₘ is a pixel width of the medium-sized pomelo fruit, and f is a focal length of the camera.

The principle of appearance image segmentation and matching is as follows:

A pomelo fruit area is extracted from the appearance image for the image segmentation. The difference between the pomelo fruit and a background in the image is significant, thus, a threshold segmentation method can be used to capture the pomelo fruit area. A dual channel fusion processing has a higher segmentation accuracy. Referring to FIG. 2, the appearance image of the pomelo fruit is converted from a RGB color space to a HSI color space, wherein a hue H channel can accurately extract a darker contour area of the pomelo fruit (a in FIG. 2), while an I channel can accurately extract a highlight and other bright areas on a surface of the pomelo fruit (c in FIG. 2). The hue H channel and the brightness I channel are extracted and the threshold segmentation are performed on the hue H channel and the brightness I channel separately. The hue H channel and the brightness I channel after the threshold segmentation are superimposed to form a complete pomelo fruit area (b in FIG. 2). The pixel heights of the pomelo fruit in the appearance image are unified using bilinear interpolation.

The principle of X-ray image segmentation and matching is as follows:

There are significant differences in grayscale values of flesh, peel, and the background in the X-ray image. Similarly, the threshold segmentation can accurately obtain the whole pomelo fruit and a flesh area in the X-ray image. For facilitating the edible rate measurement, the bilinear interpolation is also used to unify the pixel heights of the pomelo fruit in both the X-ray image and the appearance image.

### Part III, Volume Algorithm

A volume measurement algorithm is as follows.

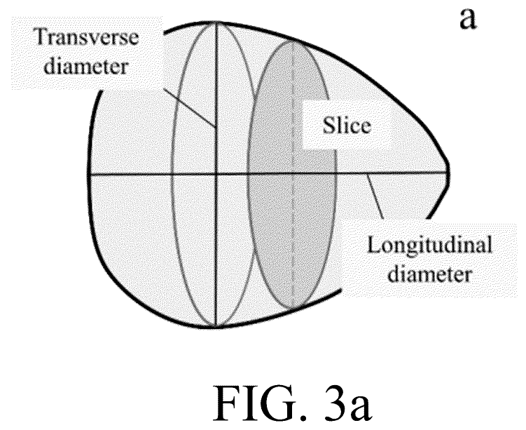

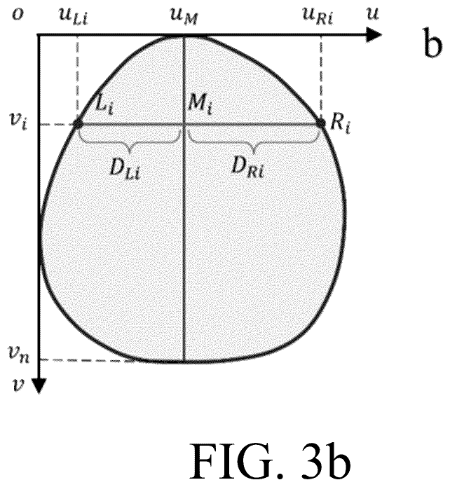

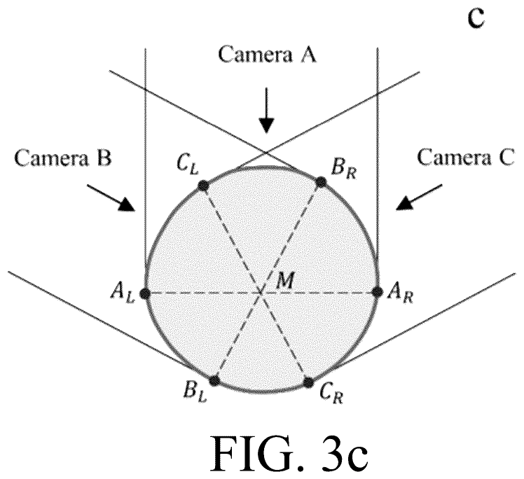

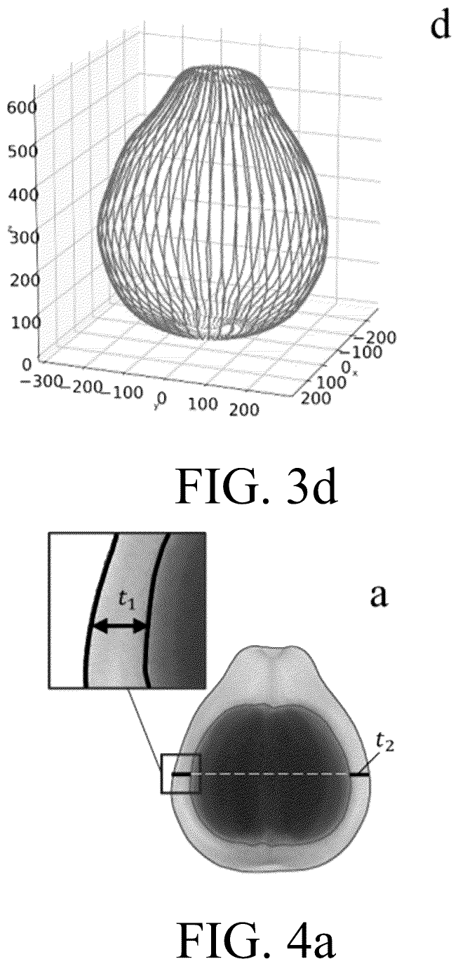

A slice integration method based on the principle of calculus is a commonly-used method for measuring volumes of fruits in machine vision. The pomelo fruit can be divided into several thin slices along a longitudinal direction thereof, as shown in FIG. 3a. A contour of each slice is a closed curve that approximates a circle. A volume of each slice can be calculated, and a total volume of the whole pomelo fruit can be obtained by adding the volumes of all the slices up. Due to the fact that each slice of the pomelo fruit is not a standard circle, the detection result is not accurate enough.

Reference for the slice integration method:
M. Omid, M. Khojastehnazhand, and A. Tabatabaeefar, “Estimating volume and mass of citrus fruits by image processing technique,” Journal of Food Engineering, vol. 2, pp. 315-321, 2010.
S. Jana, R. Parekh, and B. Sarkar, “A De novo approach for automatic volume and mass estimation of fruits and vegetables,” Optik, vol. 163443, 2020.

In this embodiment, an improved volume measurement algorithm is used to establish a three-dimensional model of the pomelo fruit.

Based on the idea of the slice integration, a new slice contour fitting method is proposed to establish a new method for measuring the volume of the pomelo fruit. As shown in FIG. 3b, the pomelo fruit area of the appearance image is divided into n slices from top to bottom. To achieve fitting of the three appearance images in a three-dimensional space, it is necessary to determine a baseline in the three images. Several top points of a stem of the pomelo fruit are selected and an average u_{M} of horizontal coordinates of the top points are calculated. A vertical line below a point (u_{M}, 0) is used as the baseline. A distance D_{Li} between a left endpoint Lᵢ of a i-th slice and the baseline, and a distance D_{Ri} between a right endpoint Rᵢ and the baseline are calculated. Six endpoints namely A_{L}, B_{L} A_{R}, B_{R}, C_{L} and C_{R}, can be extracted from the slices of the three appearance images of each pomelo fruit. A position of each endpoint in the slice is determined in a polar coordinate by using a position M in the baseline as a pole, the distance between the corresponding endpoint and the baseline as a polar diameter, an angle between the camera and the horizontal plane as a polar angle, as shown in FIG. 3c. Based on these endpoints, different fitting methods can be used to determine a slice outline of the pomelo fruit. In this embodiment, four methods are selected as follows.

(i) Mean radius circle fitting method: Fitting the slice into a circle by taking the average distance between the six endpoints and the pole as a radius and the pole as a center of the circle.

(ii) Least squares circle fitting method: Fitting the slice into a circle using a least squares method based on the known endpoints.

(iii) Least squares ellipse fitting method: Fitting the slice into an ellipse using the least squares method based on the known endpoints.

References for the least squares circle fitting algorithm and the least squares ellipse fitting algorithm:
W. Gander, G. H. Golub and R. Strebel, Least-squares fitting of circles and ellipses, BIT 4 (1994) 558-578.

(iv) Spline curve interpolation fitting method: Interpolating and fitting the known endpoints using a B-spline curve to obtain a closed curve as the slice outline.

The spline curve interpolation fitting algorithm can refer to the following references:
J. Fang and C. Hung, An improved parameterization method for B-spline curve and surface interpolation, Computer aided design 6 (2013) 1005-1028.

The four contour fitting methods are used to explore the impact of different fitting methods on the volume measurement result based on original image resolution with 1000×750 pixels: the mean radius circle fitting method, the least squares circle fitting method, the least squares ellipse fitting method, and the B-spline curve fitting method. The volume measurement results of a test set of the pomelo fruit are shown in Table 1. The volume measurement results obtained by the four fitting methods respectively have a R² being greater than 0.980, indicating that the volume measurement method based on multiple contours in this embodiment has a high measurement accuracy. Average relative errors of the four fitting methods are 2.15%, 2.22%, 2.09%, and 1.87%, respectively.

The B-spline curve fitting method for measuring the outer contour has the highest accuracy (MAPE=1.87%, RMSE=53.94 mL). The B-spline is a piecewise polynomial that utilizes linear combinations of basis functions which are also piecewise polynomials. Compared to the circle or the ellipse, the B-spline curve shape has a higher flexibility and can approach the real contour of the pomelo fruit more closely. The least squares ellipse fitting method has a higher accuracy than the other two types of circle fitting methods because the circle is a special case of ellipse, and a fitting ability of the circle is more limited. In addition, according to the ellipse formula, at least 5 discrete points are required to fit the ellipse using the least squares fitting method, thereby, images need to be captured from at least three perspectives. Therefore, the highest R² of the least squares ellipse fitting method is 0.985, slightly lower than that of the fitting method used in this embodiment. The volume measurement results of the mean radius circle fitting method are similar to those of the least squares circle fitting method, because a cross-sectional shape of the pomelo fruit is not an ideal circle. The fitted slice obtained by using the circle fitting method is suitable for the volume measurement with a single camera. The specific results can refer to Table 1.

TABLE 1
Results of Different Fitting Methods

| | Volume | | |
|---|---|---|---|
| Fitting methods | R² | MAPE (%) | RMSE (mL) |
| Mean radius circle fitting | 0.989 | 2.15 | 60.18 |
| Least squares circle fitting | 0.990 | 2.22 | 62.85 |
| Least squares ellipse fitting | 0.989 | 2.09 | 59.11 |
| B-spline curve fitting | 0.989 | 1.87 | 53.94 |

An outer contour three-dimensional model of the pomelo fruit can be formed by integrating the fitted slice contour along the baseline, as shown in FIG. 3d.

The formula for calculating the volume of the pomelo fruit is as follows:

$${V}_{P} = {\sum }_{i = 1}^{{H}_{P}} \frac{{N}_{i} \times {{Z}_{A}}^{3}}{{{f}_{x}}^{2} \times {f}_{y}} \qquad ( 3 )$$

Wherein H_{P} represents a number of the slices of the whole pomelo fruit, Nᵢ is a number of pixels enclosed by an i-th slice outline, and Z_{A} is the distance between the camera A and the pomelo fruit.

The outer contour three-dimensional model of the pomelo fruit determined above is used in subsequent edible rate algorithms.

### Part IV, Edible Rate Algorithm

The edible rate algorithm includes a three-dimensional modeling method (3DMM) and a grayscale and thickness fitting method (GTFM). Regarding the two algorithms, the following will be divided into two parts to discuss the advantages and disadvantages of the two above algorithms in detail.

### (i) 3DMM

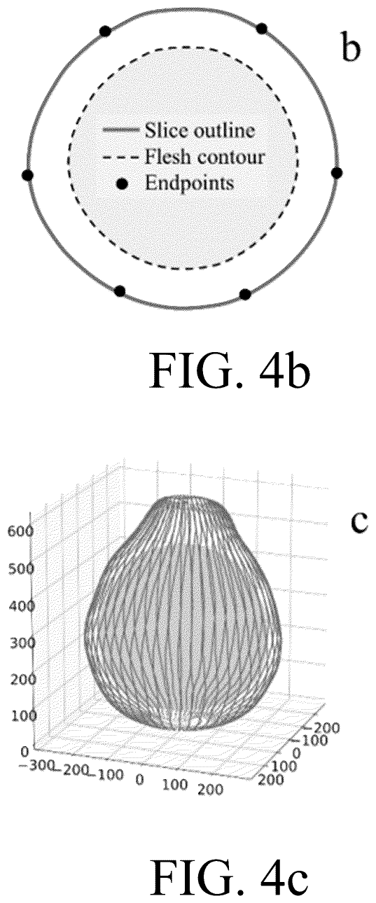

A three-dimensional model of the flesh can be further established to calculate the edible rate based on the outer contour three-dimensional model and the X-ray image established above based on the volume measurement algorithm. According to a Pascal's principle, the pomelo fruit is regarded as a closed container, and a pressure applied to the peel by the flesh is simultaneously and equally transmitted to all points of the peel during the growth process of the pomelo fruit. Therefore, a thickness of the peel in the same slice is substantially equal. In the X-ray image, the pixel thicknesses t₁ and t₂ of the peel at both ends of each slice can be obtained, as shown in FIG. 4a. The average of the pixel thicknesses t₁ and t₂ is taken as the average pixel thickness t of the slice. The fitted slice outline obtained above is contracted inward for the pixel thickness t, that is, the polar diameter of each point on the outer contour decreases by t to obtain a new contour line which can be used as a flesh contour in the slice, as shown in FIG. 4b.

By integrating the slices with the flesh contours obtained using the above methods along the baseline, a three-dimensional model of the pomelo fruit with the flesh can be obtained, as shown in FIG. 4c. Through this model, a flesh volume V_{F} can be calculated as follows:

$${V}_{F} = {\sum }_{i = 1}^{{H}_{F}} \frac{{M}_{i} \times {{Z}_{A}}^{3}}{{{f}_{x}}^{2} \times {f}_{y}} \qquad ( 4 )$$

Wherein H_{F} represents the number of the slices with the flesh contours, Mᵢ is a number of pixels enclosed by a flesh contour of an i-th slice with the flesh contour, Z_{A} is the distance between the camera A and the pomelo fruit, and fₓ and f_{y} are the focal lengths of the camera A. Due to the specific measurement of a weight of the whole pomelo fruit in commercial pomelo fruit product lines, the edible rate can be directly calculated using the weight of the whole pomelo fruit and a weight of the flesh of the pomelo fruit. According to the 3DMM, the edible rate E₁ of the pomelo fruit is obtained as shown in Formula (5):

$${E}_{1} = \frac{{V}_{F} \cdot {ρ}_{F}}{{m}_{P}} \qquad ( 5 )$$

Wherein ρ_{F} is a density of the flesh and m_{P} is the weight of the whole pomelo fruit.

It should be noted that in the present disclosure and subsequent formulas, the density ρ_{F} is the average value analyzed based on destructive sampling of multiple samples.

### (ii) Grayscale and Thickness Fitting Method (GTFM)

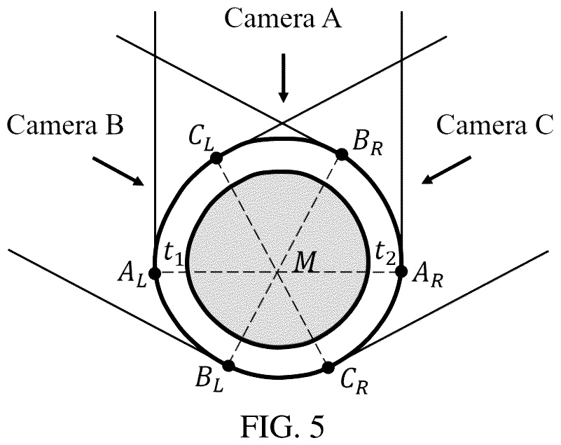

In the X-ray image, the pixel thicknesses t₁ and t₂ of the peel at both ends of each slice can be obtained, as shown in FIG. 5. The average of the pixel thicknesses t₁ and t₂ is taken as the average pixel thickness t of the slice. The fitted slice outline obtained above is contracted inward for the pixel thickness t, that is, the polar diameter of each point on the outer contour decreases by t to obtain a new contour line obtained which can be used as the flesh contour of the slice, as shown in FIG. 5. A flesh area in the three-dimensional model of the pomelo fruit can be obtained using the 3DMM.

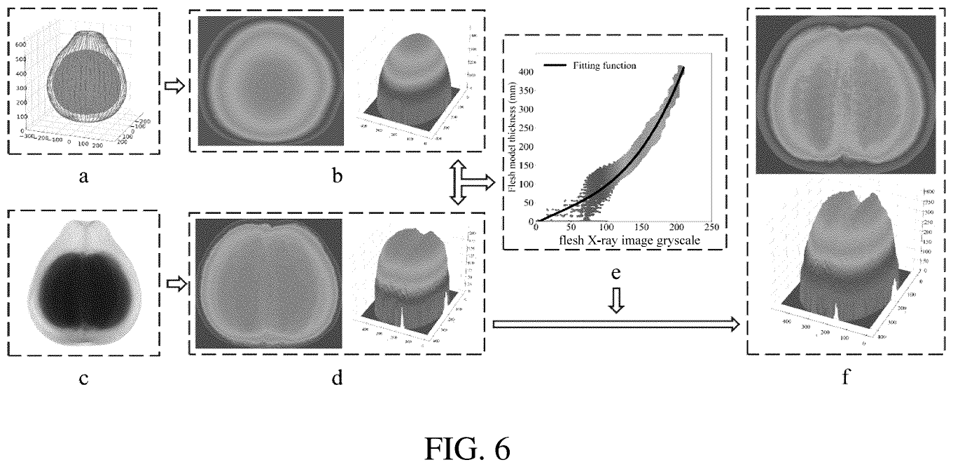

Using a transverse diameter as an x-axis direction and a longitudinal diameter a y-axis directions, a flesh model thickness image can be established by projecting the flesh area (a as shown in FIG. 6) in the three-dimensional model of the pomelo fruit along the X-ray imaging direction, as shown in FIG. 3b. A value of each point in the two-dimensional flesh model thickness image represents the thickness of the flesh at that point along the X-ray imaging direction. The flesh area is extracted from the X-ray image as a flesh X-ray image, as shown in FIG. 6 (d). The flesh model thickness image or the flesh X-ray image has a point-to-point correspondence with the actual flesh. Therefore, an appropriate fitting method can be used. A fitting function between a thickness of a certain point in the flesh model thickness image and a grayscale of the certain point in the flesh X-ray image is shown in FIG. 6 (e). The flesh X-ray image can be corrected using a fitting function to obtain a corrected flesh thickness image, as shown in FIG. 6 (f). At this time, a value of each point in the corrected flesh thickness image is a fitted flesh thickness of that point. The sum of the values of all points in the corrected flesh thickness image is the optimized flesh volume.

According to the GTFM, the final corrected edible rate E_{c} can be obtained as follows:

$${E}_{2} = \frac{{\sum }_{i}^{H} {\sum }_{j}^{W} f ( {g}_{ij} ) \cdot {ρ}_{F}}{{m}_{P}} \qquad ( 6 )$$

wherein ρ_{F} is the density of the flesh, m_{P} is the weight of the whole pomelo fruit; gᵢⱼ is a grayscale value of a point at an i-th row and a j-th column in the flesh X-ray image, H and W are respectively a height and a width of the X-ray image of the flesh, and f is the fitting function between the thickness and the grayscale.

For the fitting function between the thickness and the grayscale, a linear function, a polynomial function, and an exponential function are used to establish the fitting relationship between the thickness of a certain point in the flesh model thickness image and the grayscale of the certain point in the flesh X-ray image. The measurement results of the edible rate using the above three fitting functions are shown in Table 2. The difference between the measurement accuracy of the edible rate using the above fitting functions is small, and the R² are all greater than 0.90. The fitting method using a cubic polynomial fitting function has the highest accuracy (R²=0.923, RMSE=2.85%). The pomelo flesh is cut into rectangular shapes of different thicknesses and X-ray images are collected to analyze the functional relationship between the thickness and the grayscale. The R² of the linear function, the exponential function, and a logarithmic function are all greater than 0.970.

References for the linear function fitting algorithm and the polynomial function fitting algorithm:
L. Pothuaud, P. Carceller and D. Hans, Correlations between grey-level variations in 2D projection images (TBS) and 3D microarchitecture: Applications in the study of human trabecular bone microarchitecture, BONE 4 (2008) 775-787.

Reference for the exponential function fitting algorithm:
J. Hu, L. Liang, X. Liu and H. Dai, Research on Penetrated Thickness and Gray Model of Radiographic Digital Imaging, Acta Optica Sinica 10 (2021) 222-227.

TABLE 2
Measurement results of the edible rate using different fitting
functions f between the grayscale and the thickness

| | Edible rate (n = 180) | | |
|---|---|---|---|
| Fitting methods | R² | MAPE (%) | RMSE (%) |
| Linear fitting | 0.913 | 4.01 | 3.50 |
| Quadratic polynomial fitting | 0.910 | 3.73 | 3.08 |
| Cubic polynomial fitting | 0.923 | 3.38 | 2.85 |
| Fourth-order polynomial | 0.922 | 3.34 | 2.89 |
| fitting | | | |
| Exponential function fitting | 0.907 | 3.77 | 3.12 |

From the above records, it can be seen that, based on the relationship between the grayscale and the thickness in the X-ray images of the object, any algorithm in this field can be used and the accuracy is relatively high.

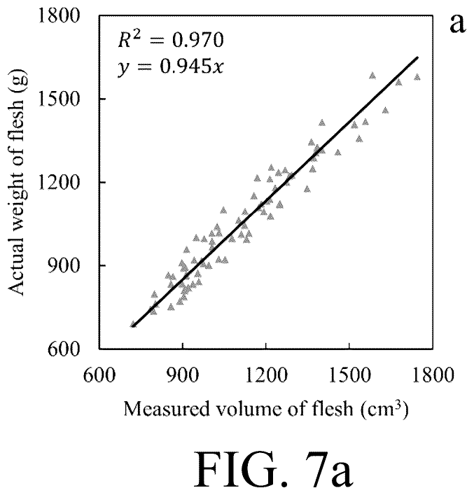

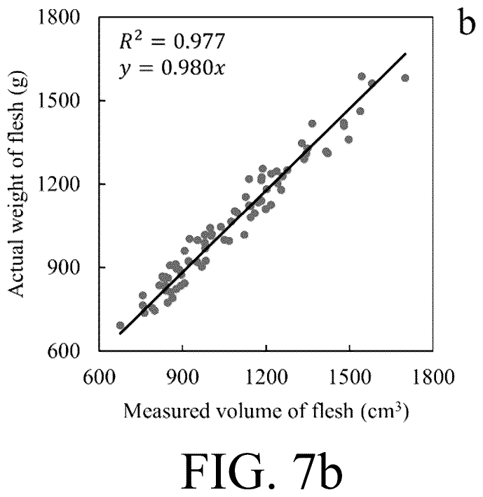

The 3DMM and the GTFM based on the 3DMM are respectively used to calculate measured flesh volumes of a training set, and the relationship between the measured flesh volume and an actual flesh weight is shown in FIGS. 7a and 7b.

FIG. 7a shows the relationship between the measured flesh volume obtained by the 3DMM and the actual flesh weight.

FIG. 7b shows the relationship between the measured flesh volume obtained by the GTFM and the actual flesh weight.

The R² between the measured flesh volume obtained by 3DMM and the actual flesh weight is 0.970, and the R² between the measured flesh volume obtained by the GTFM and the actual flesh weight is 0.977. There is a strong linear relationship between the flesh volume and the flesh weight, indicating that the density of the pomelo fruit is approximately constant. The flesh density calculated by the 3DMM is 0.945 g/cm³ and the flesh density calculated by the GTFM is 0.980 g/cm³.

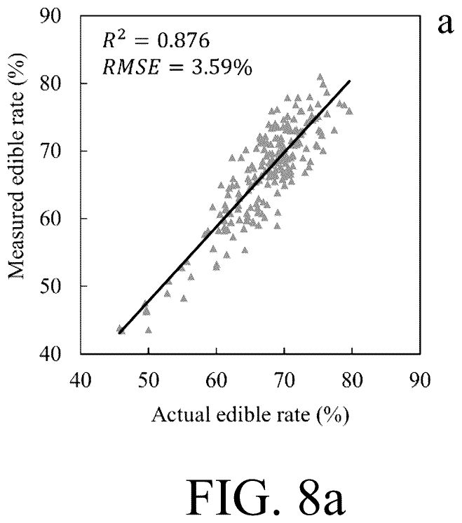

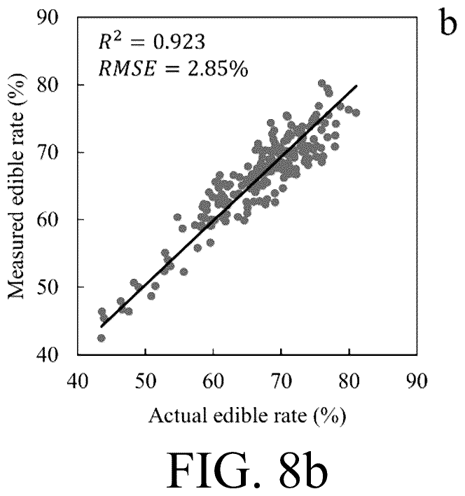

In addition, the flesh density calculated by the 3DMM is lower, which is in line with expectations. From this, it can be seen that the flesh volume measured by the GTFM is more accurate. According to Formulas (5) and (6), the edible rate measurement results of the pomelo fruit using the two methods are shown in FIG. 8a and FIG. 8b.

FIG. 8a shows the edible rate measurement results obtained by the 3DMM.

FIG. 8b shows the edible rate measurement results obtained by the 3DMM.

The R² between the measured edible rate obtained by the 3DMM and the actual edible rate is 0.876, and the RMSE is 3.59%. The GTFM (R²=0.923, RMSE=2.85%) selects the cubic polynomial function as the fitting function, thus, the GTFM has a higher accuracy than the 3DMM.

The flesh X-ray image based on the GTFM contains information about a central cavity of the pomelo fruit and a surface undulation of the flesh, which significantly improves the detection accuracy. The above results indicate that the GTFM has a better measurement accuracy, therefore the GTFM is selected for subsequent analysis. However, the GTFM also has certain measurement errors, which may be caused by the following reasons. On the one hand, since only one X-ray image instead of a Xay-CT is used, it is impossible to obtain the thickness of the peel at all positions of the pomelo fruit. Therefore, it can only assume that the thickness of the peel is substantially equal in the same slice everywhere, which may differ from the real slice. On the other hand, the X-ray flesh area is the result of the joint projection of the peel, the flesh, and the septal membrane. However, since the flesh and the septal membrane are closed to each other and are hard to separate from each other, the influence of the septal membrane on the measurement of the edible rate cannot be eliminated through image processing.

### SUMMARY

The present disclosure a rapid detection algorithm for the volume and the edible rate of the pomelo fruit based on image fusion, which solves the problems of insufficient accuracy and efficiency in the pomelo fruit quality detection in practical applications. In the algorithm, the appearance image and the X-ray image information of the pomelo fruit are combined, the outer contour of the pomelo fruit is fitted based on the B-spline curve interpolation to establish the three-dimensional model of the pomelo fruit to calculate the volume based on the model (R²=0.989, MAPE=1.87%). Comparing the two edible rate measurement methods, namely the 3DDM and the GTFM, experiments show that the GTFM is more accurate in measuring the edible rate (R²=0.923, RMSE=2.85%). The flesh volume measured by the GTFM has a strong linear relationship with the actual weight, with the R² being 0.977, and the flesh density can be calculated as 0.980 g/cm³. The method for measuring volume and edible rate proposed in this embodiment, taking the pomelo fruit as an example, is also applicable to the sorting of other fruits, providing an effective technical approach for non-destructive detection of the volume and the edible rate of the fruit.

Referring to FIG. 1, the present disclosure further provides a device for measuring an edible rate of a pomelo fruit based on machine vision and X-ray imaging. Using the above method for measuring an edible rate of a pomelo fruit based on machine vision and X-ray imaging, the device includes a conveyor belt, a RGB camera, a camera bracket, and a X-ray detection apparatus. The RGB camera is fixed on the camera bracket for adjusting a position of the pomelo fruit. The conveyor belt is used for transporting the pomelo fruit. The X-ray detection apparatus is equipped with a radiation source for X-ray detection of the pomelo fruit.

The embodiments in the present disclosure are described in a progressive method, with each embodiment focusing on the differences from other embodiments. The same and similar parts between each embodiment can be referred to each other.

It is understandable that the above-mentioned technical features may be used in any combination without limitation. The above descriptions are only the embodiments of the present disclosure, which do not limit the scope of the present disclosure. Any equivalent structure or equivalent process transformation made by using the content of the description and drawings of the present disclosure, or directly or indirectly applied to other related technologies in the same way, all fields are included in the scope of patent protection of the present disclosure.

What is claimed is:

1. A method for measuring an edible rate of a pomelo fruit based on machine vision and X-ray imaging, comprising:
step 1, collecting an appearance image of a pomelo fruit;
step 2, performing image segmentation on the appearance image of the pomelo fruit and removing a background image of the pomelo fruit;
step 3, slicing the appearance image of the pomelo fruit, determining an endpoint on each slice of the pomelo fruit, and determining a slice outline using a B-spline curve interpolation fitting method based on the endpoint;
step 4, based on the slice outline, establishing a fresh model thickness image by projecting a flesh area in a three-dimensional model of the pomelo fruit along a X-ray imaging direction, with a transverse diameter and a longitudinal diameter of the slice as an x-axis direction and a y-axis direction, respectively;
step 5, a value of each point in the flesh model thickness image representing a flesh thickness of the corresponding point along the X-ray imaging direction.

2. The method according to claim 1, wherein before the collecting an appearance image of a pomelo fruit, a Zhang's calibration is performed using a RGB camera, and a distortion correction is performed based on an intrinsic parameter, an extrinsic parameters, and a distortion coefficient of the camera.

3. The method according to claim 1, wherein the distortion correction is performed based on a geometric relationship to obtain an actual width D of the pomelo fruit as follows:
$$D = \frac{w}{d} \sqrt{{w}^{2} + {d}^{2}} \qquad ( 1 )$$
wherein w is a pixel width of the pomelo fruit in the appearance image; d is a distance between the pomelo fruit and the camera, and a corrected distance d between another pomelo fruit and the camera is defined as:
$$d = {d}_{m} + \frac{0.5 \times {d}_{m} ( {w}_{m} - w )}{f} \qquad ( 2 )$$
wherein wₘ is a distance between a medium-sized pomelo fruit and the camera, dₘ is a pixel width of the medium-sized pomelo fruit, and f is a focal length of the camera.

4. The method according to claim 1, wherein in the step 2, a threshold segmentation method is used to capture a pomelo fruit area, and the step 2 comprises:
and a dual channel fusion processing method is used to converting the appearance image of the pomelo fruit from a RGB color space to a HSI color space using a dual channel fusion processing method;
extracting a hue H channel and a brightness I channel;
performing threshold segmentation on the hue H channel and the brightness I channel respectively; and
superimposing the hue H channel and the brightness I channel after the threshold segmentation to form the pomelo fruit area.

5. The method according to claim 1, wherein in step 4, pixel thicknesses t₁ and t₂ of peel of the pomelo fruit at both ends of each slice is obtained in an X-ray image, and an average of the pixel thicknesses t₁ and t₂ is taken as an average pixel thickness t of the slice; the slice outline is contracted inward for the pixel thickness t, which indicates that a polar diameter of each point on the outer contour is decreased by t to obtain a new contour line which is used as a flesh contour in the slice.

6. A device for measuring an edible rate of a pomelo fruit based on machine vision and X-ray imaging, using the method for measuring an edible rate of a pomelo fruit based on machine vision and X-ray imaging in claim 1, wherein the device comprises a conveyor belt, a RGB camera, a camera bracket, and a X-ray detection apparatus; the RGB camera is fixed on the camera bracket for adjusting a position of the pomelo fruit; the conveyor belt is used for transporting the pomelo fruit; and the X-ray detection apparatus is equipped with a radiation source for X-ray detection of the pomelo fruit.

7. A device for measuring an edible rate of a pomelo fruit based on machine vision and X-ray imaging, using the method for measuring an edible rate of a pomelo fruit based on machine vision and X-ray imaging in claim 2, wherein the device comprises a conveyor belt, a RGB camera, a camera bracket, and a X-ray detection apparatus; the RGB camera is fixed on the camera bracket for adjusting a position of the pomelo fruit; the conveyor belt is used for transporting the pomelo fruit; and the X-ray detection apparatus is equipped with a radiation source for X-ray detection of the pomelo fruit.

8. A device for measuring an edible rate of a pomelo fruit based on machine vision and X-ray imaging, using the method for measuring an edible rate of a pomelo fruit based on machine vision and X-ray imaging in claim 3, wherein the device comprises a conveyor belt, a RGB camera, a camera bracket, and a X-ray detection apparatus; the RGB camera is fixed on the camera bracket for adjusting a position of the pomelo fruit; the conveyor belt is used for transporting the pomelo fruit; and the X-ray detection apparatus is equipped with a radiation source for X-ray detection of the pomelo fruit.

9. A device for measuring an edible rate of a pomelo fruit based on machine vision and X-ray imaging, using the method for measuring an edible rate of a pomelo fruit based on machine vision and X-ray imaging in claim 4, wherein the device comprises a conveyor belt, a RGB camera, a camera bracket, and a X-ray detection apparatus; the RGB camera is fixed on the camera bracket for adjusting a position of the pomelo fruit; the conveyor belt is used for transporting the pomelo fruit; and the X-ray detection apparatus is equipped with a radiation source for X-ray detection of the pomelo fruit.

10. A device for measuring an edible rate of a pomelo fruit based on machine vision and X-ray imaging, using the method for measuring an edible rate of a pomelo fruit based on machine vision and X-ray imaging in claim 5, wherein the device comprises a conveyor belt, a RGB camera, a camera bracket, and a X-ray detection apparatus; the RGB camera is fixed on the camera bracket for adjusting a position of the pomelo fruit; the conveyor belt is used for transporting the pomelo fruit; and the X-ray detection apparatus is equipped with a radiation source for X-ray detection of the pomelo fruit.
